(19) United States
(12) Patent Application Publication
(10) Pub. No.: US 2004/0181072 A1
(43) Pub. Date: Sep. 16, 2004

(54) Method for enhancing the productivity of vanadium antimony oxide catalysts

(76) Inventors: James F. Brazdil, Glen Ellyn, IL (US); Fernando Antonio Pessoa Cavalcanti, Lafayette Hill, PA (US)

Correspondence Address: CAROL WILSON, BP AMERICA INC., MAIL CODE 5 EAST, 4101 WINFIELD ROAD, WARRENVILLE, IL, 60555, US

(21) Appl. No.: 10/794,140

(22) Filed: Mar. 5, 2004

Related U.S. Application Data
(62) Division of application No. 10/306,298, filed on Nov. 27, 2002.

(51) Int. Cl. C07D 213/84; C07D 307/89

(52) U.S. Cl. 546/286; 502/312; 502/325; 549/248; 558/325

(57) ABSTRACT

A process for the manufacture of an improved vanadium antimony oxide oxidation or ammoxidation catalyst which comprises heat treating the catalyst at a temperature above 780° C. in the presence of an oxygen enriched environment. Such catalysts are useful in processes for the ammoxidation of a C₃-C₅ paraffinic hydrocarbon to its corresponding α-β-unsaturated nitrile, the ammoxidation of propylene with NH₃ and oxygen to acrylonitrile, the ammoxidation of methylpyridine with NH₃ and oxygen to make cyanopyridine, the ammoxidation of m-xylene with NH₃ and oxygen to make isophthalonitrile, and the oxidation of o-xylene to make phthalic anhydride.

### BACKGROUND OF THE INVENTION

1. Field of the Invention

The present invention is directed to a process for the manufacture of vanadium antimony oxide catalyst useful in the ammoxidation of a C₃-C₅ paraffinic hydrocarbon to its corresponding α-β-unsaturated nitrile ammoxidation of propylene with NH₃ and oxygen to acrylonitrile, ammoxidation of methylpyridine with NH₃ and oxygen to make cyanopyridine, the ammoxidation of m-xylene with NH₃ and oxygen to make isophthalonitrile and the oxidation of o-xylene to make phthalic anhydride. In particular, the present invention is directed to a process for the manufacture of a vanadium antimony oxide based catalyst useful in the ammoxidation of propane to acrylonitrile. More specifically, the instant invention relates to the preparation of a vanadium antimony based catalyst comprising an improved method for the heat treatment of the catalyst, which improves the productivity of such catalysts.

2. Description of the Prior Art

Commercial processes for the production of acrylonitrile employ propylene as a feedstock. However, because of the price differential between propylene and propane, an economic incentive exists for the development of a commercial process for the ammoxidation of propane to acrylonitrile. The development of such a process depends upon a viable catalyst useful for the conversion of propane to acrylonitrile.

Vanadium antimony type catalysts useful in the ammoxidation of propane to acrylonitrile along with various methods of making such catalysts are taught in the following U.S. Pat. Nos. 6,083,869; 5,994,259; 5,866,502; 5,498,588; 5,3.32,855; 5,214,016; 5,008,427; 5,258,543; 4,788,317; 4,784,979; 4,746,641; 3,860,534; and 3,681,421.

U.S. Pat. Nos. 5,675,057 and 5,696,047 disclose the preparation of vanadium antimony type catalysts wherein the catalyst is heat treated twice prior to use. The catalyst is first heat treated at a temperature above 750° C., followed by a second heat treatment, which is at least 500° C. and at least 50° C. below the first heat treatment temperature.

An improvement to the heat-treating regimen of U.S. Pat. Nos. 5,675,057 and 5,696,047 has now been discovered.

### SUMMARY OF THE INVENTION

The present invention is directed to a vanadium antimony oxide catalyst comprising vanadium, antimony, at least one of tin, titanium, iron, chromium and gallium, and optionally at least one element selected from the group consisting of lithium, magnesium, calcium, strontium, barium, cobalt, chromium, gallium, nickel, zinc, germanium, niobium, zirconium, molybdenum, tungsten, copper, tellurium, tantalum, selenium, bismuth, cerium, indium, arsenic, boron, aluminum, and manganese, wherein the relative ratios of these elements are represented by the following general formula:

V₁SbₘAₐD_{d}Oₓ

wherein A is at least one of Ti, Sn, Fe, Cr, and Ga.

D when present is at least one of Li, Mg, Ca, Sr, Ba, Co, Ni, Zn, Ge, Nb, Zr, Mo, W, Cu, Te, Ta, Se, Bi, Ce, In, As, B, Al, and Mn,

and wherein m is between about 0.5 to about 10, a is between about 0.01 to about 10, d is 0 to about 10, and x is determined by the oxidation state of the cations present; wherein the catalyst is manufactured in a process comprising heat treating the catalyst at a calcination temperature above 780° C. in the presence of an oxygen enriched environment;

In another embodiment of the instant invention the catalyst is further heat treating the catalyst at an effective temperature which is at least 500° C. and at least 50° C. below said calcination temperature.

Further embodiments of the present invention are directed to a process for producing the above vanadium antimony oxide catalyst as well as a process for the manufacture of acrylonitrile from a hydrocarbon selected from the group consisting of propylene, propane and mixtures thereof, the process comprising reacting the hydrocarbon with ammonia and oxygen in the presence of such vanadium antimony oxide catalyst.

### DETAILED DESCRIPTION OF THE INVENTION

Catalyst Composition

The vanadium antimony oxide catalysts described herein comprise vanadium, antimony, at least one of tin, titanium, iron, chromium and gallium, and optionally at least one element selected from the group consisting of lithium, magnesium, calcium, strontium, barium, cobalt, nickel, zinc, germanium, niobium, zirconium, molybdenum, tungsten, copper, tellurium, tantalum, selenium, bismuth, cerium, indium, arsenic, boron, aluminum, and manganese, wherein the relative ratios of these elements are represented by the following general formula:

V₁ SbₘAₐD_{d}Oₓ

wherein A is at least one of Ti, Sn, Fe, Cr, and Ga.

D when present is at least one of Li, Mg, Ca, Sr, Ba, Co, Ni, Zn, Ge, Nb, Zr, Mo, W, Cu, Te, Ta, Se, Bi, Ce, In, As, B, Al, and Mn,

0.5≦m≦10,

0<a≦10,

0≦d≦10, and

x is determined by the oxidation state of the cations present.

A preferred catalyst formulation, when applied to a process of manufacturing acrylonitrile or methacrylonitrile by catalytic reaction in the vapor phase of a paraffin selected from propane and isobutane with molecular oxygen and ammonia by catalytic contact of the reactants in a reaction zone, comprises vanadium, antimony, iron, molybdenum, arsenic, at least one of tin, titanium, chromium and gallium, and at least one other promoter element selected from the group consisting of lithium, magnesium, calcium, strontium, barium, cobalt, chromium, gallium, nickel, zinc, germanium, niobium, zirconium, molybdenum, tungsten, copper, tellurium, tantalum, selenium, bismuth, cerium, indium, arsenic, boron, aluminum, and manganese, wherein the relative proportions of these elements are represented by the following formula:

VₐSb_{b}A_{c}Fe_{d}DₑQ_{f}R_{g}Oₓ

where

A is at least one of Ti, Sn, Cr, and Ga.

D is at least one of Li, Mg, Ca, Sr, Ba, Co, Ni, Zn, Ge, Nb, Zr, W, Cu, Te, Ta, Se, Bi, Ce, In, B, Al, and Mn

Q is Mo

R is As

a is 1

0.8≦b≦4

0.01≦c≦2

0.01≦d≦2

0≦e≦2

0≦f≦0.01 and more preferably 0<f<0.0045

0≦g≦0.1

x is determined by the oxidation state of the cations present. In the above-described catalysts preferably “A” is Sn and Ti.

The above-described catalysts may be unsupported or supported on any suitable carrier. Examples of suitable carriers are silica, alumina, silica alumina, zirconia and the like.

Representative catalyst formulations made by the process of the instant invention include:

VSb_{1.5}Sn_{0.2}Ox

VSb₂Ti_{0.5}Ox

VSb_{1.4}Sn_{0.1} Fe_{0.2}Ox

VSb₅Cu₂Ox

VSb₂Cr_{0.2}Ga_{0.5}OX

VSb₃Sn₂Fe_{0.6}Mo_{0.05}Ox

VSb₂Mg_{0.5}B₁Ox

VSb₆Co₁ Cr_{0.6}Cu_{0.5}Ox

VSb_{1.5}Ti_{0.3}Li_{0.5}Ox

VSb_{1.7}Mn_{0.6}Mo_{0.1}Ox

VSb₈Ti₅Cr₁Cu_{0.5}Ox

Catalyst Preparation

The production of the vanadium antimony oxide based catalysts described herein begins with the preparation of a catalyst precursor dispersion, solution, sol, or slurry (preferably but not exclusively an aqueous dispersion, solution, sol, or slurry) comprising vanadium, antimony and other promoter elements, referred to herein as the “catalyst precursor slurry”. Optionally, the slurry may be prepared using a liquid solvent medium, which comprises an organic solvent, e.g. a liquid solvent comprising a mixture of water and an alcohol. This vanadium antimony oxide catalyst precursor slurry can be prepared by any method known in the art. Source compound for the vanadium, antimony and promoter elements are as described below.

A particularly effective method of preparation of the catalyst precursor slurry is disclosed in U.S. Pat. No. 5,866,502. This method comprises heating an aqueous mixture comprising water soluble vanadates (e.g. VO₄⁻³, VO₃⁻¹, V₂O₅) and an antimony containing compound (preferably Sb₂O₃) and, optionally, at least one compound comprising a promoter element to a temperature between 110° C. and 250° C. under autogenous pressure with agitation for a time sufficient to allow at least the slightly water soluble vanadates and the antimony containing compound to react to form the catalyst precursor slurry.

The hydrothermal reaction of the metal oxides in the aqueous solution is continued for a time period sufficient for the metal oxides to suitably react to form the catalyst precursor. The required reaction time is ultimately determined by the catalytic and physical properties of the final material obtained after calcination. Typically, the reaction is continued for between 0.5 to 100 hrs, preferably from 1 to 50 hrs, especially preferred being 1 to 10 hrs. It has been observed that shorter reaction times are required as one increases the temperature employed during the catalyst precursor formation.

An alternative method of preparation of the catalyst precursor slurry is the so-called “peroxide method” disclosed in U.S. Pat. Nos. 4,784,979 and 4,879,264. Specifically according to U.S. Pat. No. 4,784,979, the catalyst precursor slurry is prepared by first preparing a monoperoxovanadium ion, VO(O₂)⁺, by reacting a vanadium compound with an aqueous hydrogen peroxide (H₂O₂) solution, and then reacting the monoperoxovanadium ion, VO(O₂)⁺, while in aqueous solution, with an antimony compound which contains Sb having a valence of 3, thereby reducing the average valence of the vanadium to less than 5 and oxidizing antimony to a valence state of 5. At least a portion of the Sb⁺³ is so reduced, not necessarily all.

The vanadium source (i.e. the vanadate, as used herein) can be an inorganic or an organic compound of vanadium, but is usually an inorganic compound. The vanadium in the compound can have any initial valence. A partial list of such compounds includes any oxide of vanadium, such as V₂O₅, V₇O₁₃, VO, VO₂, V₂O₃, V₃O₇, etc.; any vanadium oxyhalide such VOCl₃, VOCl₂, (VO₂)Cl, VOCl, VOBr, VOBr₂, VOBr₃; any vanadium halide such as VF₃, VBr₃, VCl₂, VCl₃, VCl₄, VF₅; vanadyl sulfate; meta-vanadic acid; pyro-vanadic acid.

For the peroxide method, the vanadium compound usually used in the reaction with H₂O₂ is one of the oxides. Because of availability and cost, V₂O₅ is often the compound that is chosen to react with the hydrogen peroxide.

The antimony source (i.e. the antimony compound or antimony containing compound, as used herein) can be an organic or an inorganic compound of antimony. A partial list of such compounds includes any of the following types of compounds containing antimony having a valence of 3: any such antimony oxide such as Sb₂O₃ and Sb₂O₄; SbOCl; any such antimony halide such as SbBr₃, SbCl₃, SbF₃ and SbI₃. The preferred antimony source in these preparations is Sb₂O₃, After the vanadium and antimony reaction has taken place, compounds comprising promoter elements may be added. These includes compounds of elements such as Ti, Sn, Fe, Cu, Mg, Mo, As, Li, Ca, Sr, Ba, Co, Ni, Zn, Ge, Nb, Zr, W, Te, Ta, Se, Bi, Ce, In, B, Al, and Mn. Examples of sources of the metal promoters include nitrates, acetates, hydroxides, oxides, ammonium ion complexes, and carbonyls. A preferred promoter is iron derived from an iron containing compound (e.g. Fe₂O₃) having a BET surface area of greater than about 120 m²/gram. For iron promoted catalysts, the atomic ratio of iron to vanadium is preferably greater than 0.2. For the peroxide prep described above, compounds of some elements such as Ti that form peroxo compounds can also be added before or with the addition of the H₂O₂, but are usually most conveniently added after the vanadium and antimony compounds have reacted. Alternatively, promoter elements may be added in sol form. Alternatively, promoter elements can be added prior to the reaction of the vanadium and antimony reaction as described in U.S. Pat. No. 5,866,502 or promoter elements can be added by impregnation after drying the catalyst precursor slurry to remove water. The addition of promoter elements to the vanadium antimony oxide catalyst precursor slurry or dried catalyst precursor can be achieved by known methods in the art such as ion-exchange, solvo thermal treatment, and impregnation.

An additional alternative is to add promoter elements in sol form. For example, U.S. Pat. No. 6,087,524 discloses the preparation of tin promoted vanadium antimony oxide catalysts using tin sols (made from SnO₂.nH₂O) wherein the tin sol was dispersed in a quaternary ammonium hydroxide. Additionally, a quaternary ammonium hydroxide (e.g. tetramethyl ammonium hydroxide or tetraethyl ammonium hydroxide can be added to the catalyst slurry by itself in order to improve attrition resistance of the final catalyst. The quaternary ammonium hydroxide is added such that the molar ratio of added QAH per gram of finished catalyst is between about 0.001 and about 10, preferably between about 0.005 and about 0.5.

The catalyst can be supported on any suitable carrier. Examples of such carriers are silica, alumina, silica-alumina, and the like. A particularly attrition resistant form of the catalyst contains silica, added as silica sol. Various types of silica sol, with particle sizes of about 5 to about 100 nanometers, can be used. The silica sol may be added to the catalyst precursor slurry at any time prior to drying the catalyst precursor slurry to form the catalyst precursor. Usually, these catalytic grade silica sols have low alkali metal content, and are stabilized by ammonia. Ion exchange with resins in acid or ammonium forms can also be used to remove excess alkali or alkaline earth ions from the silica.

After making the catalyst precursor slurry as described above the precursor slurry is dried to remove water to yield a catalyst precursor, which is then calcined to produce the finished catalyst. Optionally, the catalyst precursor slurry may first be concentrated by heating the catalyst precursor slurry in order to evaporate residual quantities of water. These heat treatments can be conducted as separate operations in multiple pieces of equipment or they can be conducted in single piece of equipment wherein the temperature is increased stepwise or continuously over time. In the preparation of a fixed bed catalyst, the catalyst precursor slurry is typically dried by heating at an elevated temperature and then shaped (e.g extruded, pellitized, etc.) to the desired fixed bed catalyst size and configuration. In the preparation of fluid bed catalysts, the catalyst precursor slurry is typically spray dried to yield microspheroidal catalyst particles having particle diameters in the range from 10 to 200 microns.

Optionally, the catalyst may be washed at any one or more points in the procedure using the methods disclosed in U.S. Pat. Nos. 3,860,534 and/or 5,094,989. In one embodiment, the catalyst can be washed after the high temperature heat treatment or calcinations described below by contacting the calcined catalyst with a hydroxy compound in liquid form (usually having no carbon-to-carbon unsaturation) selected from (1) cyclohexanol, (2) cyclopentanol, (3) a monohydroxy, acyclic hydrocarbon having 1-8 C atoms, usually 1-10 C atoms, and (4) a dihydroxy, acyclic hydrocarbon having 2-4 carbon atoms, and separating as a liquid said compound from said catalyst insofar as it is present beyond the amount wetting said catalyst, and thereafter drying said catalyst. Especially useful hydroxy compounds are the monohydroxy, acyclic hydrocarbons having 1 to 8 carbon atoms, and the dihydroxy, acyclic hydrocarbons having 2 to 4 carbon atoms. Most useful are the monohydroxy, acyclic hydrocarbons having 1 to 4 carbon atoms, especially isobutanol.

Heat Treatment

The hallmark of the instant invention is the heat treatment of the catalyst, which improves the productivity of the catalyst. After the catalyst is dried the catalyst is subjected to a high temperature heat treatment or calcination in an oxygen-enriched environment. The high temperature heat treatment or calcination is conducted at a temperature of at least 600° C., preferably at least 750° C., more preferably at least 780° C. For vanadium antimony oxide catalysts used for the ammoxidation of propane a high temperature heat treatment or calcination at a temperature of at least 780° C. is preferred. The high temperature heat treatment or calcination temperatures can be as high as 1200° C. but are more preferably in the range of about 790° C. to about 1050° C. Such heat treatments were previously conducted in air. The length of the calcination period may range from 30 minutes to 12 hours, with a more typical calcinations period of 1 to 5 hours. As used herein “an oxygen enriched environment” is a gaseous environment or atmosphere having a greater oxygen (O₂) content than air. The oxygen content of such oxygen-enriched environment is typically greater than 21% by volume, preferably greater than 50% by volume, and most preferably greater than 99% by volume.

Optionally, the catalyst may be further heat treated at a temperature that is at least 500° C. and at least 50° C. below said high temperature heat treatment calcination temperature.

Processes

In another aspect of the present invention, there is provided a process for making an α,β unsaturated mononitrile selected from acrylonitrile and methacrylonitrile, by catalytic reaction in the vapor phase of a paraffin selected from propane and isobutane with molecular oxygen and ammonia and optionally a gaseous diluent, by catalytic contact of the foregoing reactants in a reaction zone with a catalyst, the feed to said reaction zone containing a mole ratio of said paraffin to NH₃ in the range of 2.5 to 16 and a mole ratio of said paraffin to O₂ in the range from 1 to 10, said catalyst having an empirical composition described above, said catalyst having been made by a method described above. The reaction temperature range can vary from 350° C. to 700° C. but is usually between 430° C. and 520° C. The average contact time can be from 0.01 to 10 seconds but is usually between 0.02 and 10 seconds and more preferably between 0.1 to 5 seconds. The pressure in the reaction zone is usually no more than 75 psia, but is preferably no more than 50 psia.

The catalyst may also be used in the ammoxidation of methylpyridine and m-xylene to cyanopyridine and isophthalonitrile or the oxidation of o-xylene to phthalic anhydride. The mole ratios of ammonia to methylpyridine and O₂ to methylpyridine are 1 to 5 and 1 to 10, respectively. The mole ratios of ammonia to m-xylene and O₂ to m-xylene are 1 to 5 and 1 to 10, respectively. In the phthalic anhydride reaction, the ratio of O₂ to o-xylene may range from 1 to 10.

The catalyst prepared by the process of the present invention may also be utilized in the ammoxidation of propylene or isobutene with ammonia and oxygen to produce acrylonitrile or methacrylonitrile. The mole ratio of ammonia to olefin may range from 1 to 5 and the mole ratio of O₂ to olefin may range from 1 to 10 in this reaction under conventional temperatures and conditions well known in the art.

The catalyst and processes described herein may be employed in any suitable reactor including fixed-bed, fluid-bed, and transport-bed reactors.

### Specific Embodiments

For purposes of illustration only, the following examples are set forth to describe the catalyst and processes of the invention:

Part I—Preparation of Catalyst

Catalyst Composition: V₁Sb_{1.4}Sn_{0.2}Ti_{0.1}Fe_{0.1}Oₓ+20% SiO₂

The catalyst was prepared using a 50 gallon temperature controlled sealed reactor vessel. The catalyst was prepared by adding 75.2 lbs. of water to the reactor followed by 84.8 lbs. of 20 wt % SnO₂ sol, 8 lbs. of a 25 wt % aqueous solution of tetramethyl ammonium hydroxide, 4.45 lbs of TiO₂, 51.2 lbs of V₂O₅, 114.8 lbs. of Sb₂O₃ and 4.5 lbs of Fe₂O₃. The reactor was closed and heated to 125° C. while the reactor contents were agitated. The reactor was maintained under these conditions for 5 hours after which it was cooled to 40° C. Once the reactor was cooled, 156.3 lbs of a 32.5 wt % SiO₂ sol was added and the resulting mixture was agitated for 0.5 hours. A portion of the resulting slurry was removed from the reactor, placed in a beaker and evaporated to near-dryness on a hot plate with constant stirring. It was then dried in an oven at 120° C. The dried material was heat treated at 325° C. for 3 hours then crushed and sieved, and particles between 20 and 35 mesh in size were collected for further heat treatment.

Part II—Catalyst Heat Treatments

Four samples of V₁Sb_{1.4}Sn_{0.2}Ti_{0.1}Fe_{0.1}Oₓ+20% SiO₂ catalyst prepared as set forth in Part 1 above were subject to two heat treatment steps. Comparative Examples A and B were both calcined in air at 820° C. for 3 hours. Subsequently, Comparative Example A was post-calcined at 650° C. for 3 hours in air while Comparative Example B was post-calcined at 650° C. for 3 hours in oxygen (greater than 99% O₂). Examples 1 and 2 underwent the heat treatment of the instant invention. Examples 1 and 2 were both calcined in oxygen at 820° C. for 3 hours. Subsequently, Example 1 was post-calcined at 650° C. for 3 hours in air while Example 2 was post-calcined at 650° C. for 3 hours in oxygen. The calcinations were done in a muffle furnace. The heat treatments of the catalyst are summarized in Table I.

The heat treated (calcined) catalysts were subsequently washed with isobutanol, then dried at 120° C. before use.

Part III—Catalyst Testing

The catalysts prepared in Part I above were tested in a fixed-bed micro-reactor made of 0.25 inch O.D. titanium tubing. The reactor is equipped with a preheat leg and is immersed in a temperature controlled molten salt bath. The feed is fed to the catalyst for one hour before collection of product, unless otherwise noted; the runs of each example last 30-60 minutes during which the product is collected for analysis The molar ratios of the feed compositions, reaction temperatures and contact times for the tests are listed in Table 2 below. All tests were conducted at a reactor temperature and pressure of 480° C. and 15 psig. Product analysis was done with two gas chromatographs. One was fitted with a packed Carbowax on Carbopak column to determine nitriles in liquids collected in an ice-cooled oxalic acid scrubber. The other was fitted with molecular sieve and silicone oil columns for analysis of fixed gases and light hydrocarbons in the feed and effluent gas streams. Ammonia and HCN were determined by titration. Acrylonitrile Selectivity is defined as the ratio of moles of acrylonitrile made to propane converted expressed in percent. Acrylonitrile Productivity is defined as the weight of acrylonitrile produced per unit weight of catalyst per hour (wt AN/wt Catalyst/hr).

TABLE 1
Heat Treatment Summary

| Example | Temperature | Atmosphere | Temperature | Atmosphere |
|---|---|---|---|---|
| No. | 1^{st} Treatment | 1^{st} Treatment | 2^{nd} Treatment | 2^{nd} Treatment |
| A | 820° C. | air | 650° C. | air |
| B | 820° C. | air | 650° C. | oxygen |
| 1 | 820° C. | oxygen | 650° C. | air |
| 2 | 820° C. | oxygen | 650° C. | oxygen |

TABLE 2
Catalyst Performance

| | Reactor Feed | | | | |
|---|---|---|---|---|---|
| | Mixture | Contact | Propane | | |
| Ex- | C₃H₈/NH₃/ | Time | Con- | Acrylonitrile | Acrylonitrile |
| ample | O₂/N₂ | (sec) | version | Selectivity | Productivity |
| A | 3/0.8/2/2 | 2.5 | 20% | 57% | 0.066 |
| B | 3/0.8/2/2 | 2.43 | 19% | 57% | 0.065 |
| 1 | 3/0.8/2/2 | 1.21 | 19% | 56% | 0.122 |
| 2 | 3/0.8/2/2 | 1.20 | 18% | 56% | 0.123 |

Notes:
1. Contact Time is the number of seconds elapsed prior to achieving approximately 19-20% propane conversion
2. Propane Conversion is defined as the percentage of propane feed which is converted to products or by-products in the reactor.
3. Acrylonitrile Selectivity is defined as the ratio of moles of acrylonitrile produced to propane converted expressed as a percent.
4. Acrylonitrile Productivity is defined as the weight in grams of acrylonitrile produced per unit weight in grams of catalyst per hour (g AN/g Catalyst/hr).

The data presented in Table 2 illustrates that vanadium antimony oxide catalysts when heat treated/calcined at a temperature above 780° C. in an oxygen enriched environment (Examples 1 and 2) exhibit greater acrylonitrile productivity than identical catalyst which were heat treated/calcined at a temperature above 780° C. in air (Examples A and B).

It is to be understood that the subject invention is not to be limited by the exact description set forth in the examples herein. These have been provided merely to demonstrate the operability of the invention herein described. The selection of catalysts, metal sources, carbon supports, concentrations, contact times, solids loadings, feedstocks, reaction conditions, and products can be determined from the total specification disclosure herein disclosed and described, without departing from the spirit and the scope of the invention, including modifications and variations, that fall within the boundaries of the attached claims.

That which is claimed is:

1. A vanadium antimony oxide catalyst comprising vanadium, antimony, at least one of tin, titanium, iron, chromium and gallium, and optionally at least one element selected from the group consisting of lithium, magnesium, calcium, strontium, barium, cobalt, nickel, zinc, germanium, niobium, zirconium, molybdenum, tungsten, copper, tellurium, tantalum, selenium, bismuth, cerium, indium, arsenic, boron, aluminum, and manganese, wherein the relative ratios of these elements are represented by the following general formula:
V₁ SbₘAₐD_{d}Oₓ
wherein A is at least one of Ti, Sn, Fe, Cr, and Ga.
D when present is at least one of Li, Mg, Ca, Sr, Ba, Co, Ni, Zn, Ge, Nb, Zr, Mo, W, Cu, Te, Ta, Se, Bi, Ce, In, As, B, Al, and Mn,
and wherein m is between about 0.5 to about 10, a is between about 0.01 to about 10, d is 0 to about 10, and x is determined by the oxidation state of the cations present; wherein the catalyst is manufactured in a process comprising heat treating the catalyst in the presence of an oxygen enriched environment.

2. The catalyst of claim 1, wherein the heat treatment is conducted at a calcination temperature of at least 600° C.

3. The catalyst of claim 1, wherein the heat treatment is conducted at a calcination temperature of at least 750° C.

4. The catalyst of claim 1, wherein the heat treatment is conducted at a calcination temperature of at least 780° C.

5. The catalyst of claim 1, wherein the heat treatment is conducted at a calcination temperature between 780° C. and 1200° C.

6. The catalyst of claim 2, wherein the heat treatment is conducted at a calcination temperature is between 790° C. and 1050° C.

7. The catalyst of claim 1, wherein the oxygen enriched environment is greater than 21% by volume oxygen.

8. The catalyst of claim 1, wherein the oxygen enriched environment is greater than 50% by volume oxygen.

9. The catalyst of claim 1, wherein the oxygen enriched environment is greater than 99% by volume oxygen.

10. The catalyst of claim 1, wherein the catalyst is further heat treated at a temperature that is at least 500° C. and at least 50° C. below said calcination temperature.

11. The vanadium antimony oxide catalyst of claim 1, wherein the catalyst comprises vanadium, antimony, iron, molybdenum, arsenic, at least one of tin, titanium, chromium and gallium, and at least one other promoter element selected from the group consisting of lithium, magnesium, calcium, strontium, barium, cobalt, chromium, gallium, nickel, zinc, germanium, niobium, zirconium, molybdenum, tungsten, copper, tellurium, tantalum, selenium, bismuth, cerium, indium, arsenic, boron, aluminum, and manganese, wherein the relative proportions of these elements are represented by the following formula:
VₐSb_{b}A_{c}Fe_{d}DₑQ_{f}R_{g}Oₓ
where
A is at least one of Ti, Sn, Cr, and Ga,
D is at least one of Li, Mg, Ca, Sr, Ba, Co, Ni, Zn, Ge, Nb, Zr, W, Cu, Te, Ta, Se, Bi, Ce, In, B, Al, and Mn,
Q is Mo,
R is As,
a is 1,
0.8≦b≦4,
0.01≦c≦2,
0.01≦d≦2,
0≦e≦2,
0<f<0.01,
0≦g<0.1, and
x is determined by the oxidation state of the cations present.

12. The vanadium antimony oxide catalyst of claim 11, wherein 0<f<0.0045.

13. A process for making a vanadium antimony oxide catalyst, comprising heat treating the catalyst in the presence of an oxygen enriched environment, wherein said catalyst comprises vanadium, antimony, at least one of tin, titanium, iron, chromium and gallium, and optionally at least one element selected from the group consisting of lithium, magnesium, calcium, strontium, barium, cobalt, nickel, zinc, germanium, niobium, zirconium, molybdenum, tungsten, copper, tellurium, tantalum, selenium, bismuth, cerium, indium, arsenic, boron, aluminum, and manganese, wherein the relative ratios of these elements are represented by the following general formula:
V₁SbₘAₐD_{d}Oₓ
wherein A is at least one of Ti, Sn, Fe, Cr, and Ga.
D when present is at least one of Li, Mg, Ca, Sr, Ba, Co, Ni, Zn, Ge, Nb, Zr, Mo, W, Cu, Te, Ta, Se, Bi, Ce, In, As, B, Al, and Mn,
and wherein m is between about 0.5 to about 10, a is between about 0.01 to about 10, d is 0 to about 10, and x is determined by the oxidation state of the cations present.

14. The process of claim 13, wherein the heat treatment is conducted at a calcination temperature of at least 600° C.

15. The process of claim 13, wherein the heat treatment is conducted at a calcination temperature of at least 750° C.

16. The process of claim 13, wherein the heat treatment is conducted at a calcination temperature of at least 780° C.

17. The process of claim 13, wherein the heat treatment is conducted at a calcination temperature between 780° C. and 1200° C.

18. The process of claim 13, wherein the heat treatment is conducted at a calcination temperature is between 790° C. and 1050° C.

19. The process of claim 13, wherein the oxygen enriched environment is greater than 21% by volume oxygen.

20. The process of claim 13, wherein the oxygen enriched environment is greater than 50% by volume oxygen.

21. The process of claim 13, wherein the oxygen enriched environment is greater than 99% by volume oxygen.

22. The process of claim 13, wherein the catalyst is further heat treated at an effective temperature that is at least 500° C. and at least 50° C. below said calcination temperature.

23. The process of claim 13, wherein the vanadium antimony oxide catalyst comprises vanadium, antimony, iron, molybdenum, arsenic, at least one of tin, titanium, chromium and gallium, and at least one other promoter element selected from the group consisting of lithium, magnesium, calcium, strontium, barium, cobalt, chromium, gallium, nickel, zinc, germanium, niobium, zirconium, molybdenum, tungsten, copper, tellurium, tantalum, selenium, bismuth, cerium, indium, arsenic, boron, aluminum, and manganese, wherein the relative proportions of these elements are represented by the following formula:
VₐSb_{b}A_{c}Fe_{d}DₑQ_{f}R_{g}Oₓ
where
A is at least one of Ti, Sn, Cr, and Ga,
D is at least one of Li, Mg, Ca, Sr, Ba, Co, Ni, Zn, Ge, Nb, Zr, W, Cu, Te, Ta, Se, Bi, Ce, In, B, Al, and Mn,
Q is Mo,
R is As,
a is 1,
0.8≦b≦4,
0.01≦c≦2,
0.01≦d≦2,
0≦e≦2,
0<f<0.01,
0≦g<0.1, and
x is determined by the oxidation state of the cations present.

24. The process of claim 23, wherein 0<f<0.0045.

25. A process for the manufacture of acrylonitrile from a hydrocarbon selected from the group consisting of propylene, propane and mixtures thereof comprising reacting the hydrocarbon with ammonia and oxygen in the presence of a vanadium antimony oxide catalyst comprising vanadium, antimony, at least one of tin, titanium, iron, chromium and gallium, and optionally at least one element selected from the group consisting of lithium, magnesium, calcium, strontium, barium, cobalt, nickel, zinc, germanium, niobium, zirconium, molybdenum, tungsten, copper, tellurium, tantalum, selenium, bismuth, cerium, indium, arsenic, boron, aluminum, and manganese, wherein the relative ratios of these elements are represented by the following general formula:
V₁ SbₘAₐD_{d}Oₓ
wherein A is at least one of Ti, Sn, Fe, Cr, and Ga.
D when present is at least one of Li, Mg, Ca, Sr, Ba, Co, Ni, Zn, Ge, Nb, Zr, Mo, W, Cu, Te, Ta, Se, Bi, Ce, In, As, B, Al, and Mn,
and wherein m is between about 0.5 to about 10, a is between about 0.01 to about 10, d is 0 to about 10, and x is determined by the oxidation state of the cations present; wherein the catalyst is manufactured in a process comprising heat treating the catalyst in the presence of an oxygen enriched environment.

26. The process of claim 25, wherein the heat treatment is conducted at a calcination temperature of at least 600° C.

27. The process of claim 25, wherein the heat treatment is conducted at a calcination temperature of at least 750° C.

28. The process of claim 25, wherein the heat treatment is conducted at a calcination temperature of at least 780° C.

29. The process of claim 25, wherein the heat treatment is conducted at a calcination temperature between 780° C. and 1200° C.

30. The process of claim 25, wherein the heat treatment is conducted at a calcination temperature is between 790° C. and 1050° C.

31. The process of claim 25, wherein the oxygen enriched environment is greater than 21% by volume oxygen.

32. The process of claim 25, wherein the oxygen enriched environment is greater than 50% by volume oxygen.

33. The process of claim 25, wherein the oxygen enriched environment is greater than 99% by volume oxygen.

34. The process of claim 25, wherein the catalyst is further heat treated at an effective temperature that is at least 500° C. and at least 50C below said calcination temperature.

35. The process of claim 25, wherein the vanadium antimony oxide catalyst comprises vanadium, antimony, iron, molybdenum, arsenic, at least one of tin, titanium, chromium and gallium, and at least one other promoter element selected from the group consisting of lithium, magnesium, calcium, strontium, barium, cobalt, chromium, gallium, nickel, zinc, germanium, niobium, zirconium, molybdenum, tungsten, copper, tellurium, tantalum, selenium, bismuth, cerium, indium, arsenic, boron, aluminum, and manganese, wherein the relative proportions of these elements are represented by the following formula:
VₐSb_{b}A_{c}Fe_{d}DₑQ_{f}R_{g}Oₓ
where
A is at least one of Ti, Sn, Cr, and Ga,
D is at least one of Li, Mg, Ca, Sr, Ba, Co, Ni, Zn, Ge, Nb, Zr, W, Cu. Te, Ta, Se, Bi, Ce, In, B, Al, and Mn,
Q is Mo,
R is As,
a is 1,
0.8≦b≦4,
0.01≦c≦2,
0.01≦d≦2,
0≦e≦2,
0<f<0.01,
0≦g<0.1, and
x is determined by the oxidation state of the cations present.

36. The process of claim 19, wherein 0<f<0.0045.
